SEQUENCE LISTING (1 sequence)

SEQ ID NO: 1 - DNA, 4104 bp, synthetic construct
  source 1..4104: mol_type = other DNA
atggacaagaagtattccatcggcctggacataggaaccaactctgtgggttgggccgttatcaccgatgagtacaaggtgccctccaagaagttcaaagtgctgggtaacaccgataggcatagcattaagaaaaatctgatcggggcattgctgtttgattccggagaaacagcagaggctactagactcaagagaactgccaggcggcggtacactaggcgcaagaataggatctgctatctgcaagagatcttttccaacgagatggccaaggttgatgactctttcttccaccgattggaggagagttttctggtggaagaggacaaaaagcacgagcggcacccaatcttcggaaacattgttgacgaggtggcctaccacgagaaataccccaccatataccatcttcgcaaaaaactggtcgatagtacggacaaggccgaccttagacttatctacctggctcttgcccatatgatcaaatttaggggacacttcttgatcgagggagatctcaacccggacaacagtgatgtggacaagctgttcattcagctggttcagacctataaccaattgtttgaggagaaccctatcaacgcttcaggcgtggacgctaaggctatactctctgcgcggctctcaaaatcacgacggttggaaaatctcatcgctcagctcccaggcgaaaagaagaacggtctgttcggtaacctgattgccctgagtctgggcctcacaccgaatttcaagagcaacttcgacctcgcagaggacgccaagctccagctgtccaaggacacctatgatgatgacctggataatctgctcgcccagatcggggatcagtatgccgatctgtttctggccgccaaaaacctgtctgacgctatactgctgagtgacattctgcgggtgaacaccgagattacaaaggccccacttagcgccagtatgatcaaaaggtacgacgagcaccatcaggacctgacactgttgaaggccctggtgagacagcaactccccgaaaagtacaaggagatctttttcgatcaatcaaaaaacggctacgctgggtatattgacggaggcgcaagccaggaagagttctacaagttcatcaaaccgattctggagaaaatggacggcaccgaagagctgcttgtgaagctgaatagagaagacctccttaggaagcagcgcactttcgacaacgggagcatcccgcaccagatacatctcggggagctgcatgctatcttgaggaggcaggaagatttttacccgttcctgaaggacaaccgcgaaaagatcgagaagatactgacgttcaggataccctactatgtggggcctctggctcgaggaaatagccgcttcgcgtggatgactagaaaatcagaggaaacaataaccccctggaattttgaagaggtggtcgacaagggggcgtctgcccagagcttcattgagcgcatgacaaacttcgacaaaaatctgcctaacgaaaaagttctgcctaaacactctcttctgtacgagtatttcactgtttataacgaactcacaaaggtgaagtatgtgaccgaaggcatgagaaaacccgcgtttctgtctggggaacagaagaaggccatcgttgacttgctgtttaagaccaatcgcaaggtcacggtgaagcagctgaaggaggactacttcaaaaagatagagtgtttcgacagcgtggaaatctccggagtcgaagaccggtttaacgcgagtctggggacctaccacgacctgctgaagattatcaaggacaaggactttctcgacaacgaggaaaacgaggatatactggaggacatcgtgttgaccctgacgctgttcgaggatcgggagatgatcgaggagcggctgaagacttatgcgcacctgtttgatgacaaggttatgaagcagctcaagagaagacggtacaccggctggggccggctttccagaaagctcatcaatggaatcagggataaacagtctggtaagacaatcttggatttcctgaagagcgatgggttcgctaaccgcaatttcatgcagctgattcacgatgattctctcacattcaaagaagatattcagaaggcccaagtctccggacagggcgattctctgcacgaacacatcgccaatcttgcggggtcccctgccatcaaaaagggaattcttcagactgtcaaggtcgtggacgagctggtgaaagtgatgggccggcacaagcctgaaaatatagtgattgagatggcaagagaaaaccaaacaactcagaaaggccagaagaattcacgagaaaggatgaagcggatagaggaaggcattaaggagctgggctcccaaattctgaaagagcatcccgtggagaacacacagctccagaacgagaagctttacttgtactacttgcagaacggcagagacatgtacgtggatcaggagctggacatcaatcgattgtcagactatgacgtggatcacatcgttccacagagcttcctggcagacgattccatcgataacaaggtgcttactcggtccgacaagaaccgggggaagagtgataatgtccccagcgaggaggtggtgaagaagatgaagaattactggagacagttgctgaatgcgaagctcatcacccagcgcaagtttgataaccttacaaaagccgaaagagggggattgtccgaattggacaaggccggatttattaagagacagcttgtcgagacccgccagatcacaaagcacgtcgctcaaatactggattcccgcatgaataccaagtacgatgaaaacgataagctgatccgggaagtgaaggtgattaccctgaaatctaaattggtgagtgactttaggaaggatttccagttctataaagtgcgagaaataaataactatcatcacgcgcatgatgcctatctgaacgcagtcgtgggcaccgcactgataaagaaataccccgccctggagagcgagtttgtgtacggcgactataaggtgtatgacgtgagaaaaatgattgccaaatccgaacaggagatcggtaaggctactgctaaatattttttctactcaaacatcatgaactttttcaagacggaaattaccttggctaatggcgaaatccgaaaagcaccccttattgagaccaatggggaaacgggcgaaatagtgtgggataagggcagagattttgcaaccgtgaggaaagtcctcagcatgccccaagtgaacatcgtgaaaaagacagaggttcaaactgggggattctccaaagagtccattctgccgaagagaaacagtgataaactgatcgcgcgcaaaaaggactgggatccgaagaagtacgggggctttgattctcctactgttgcgtacagcgtgctcgtcgtcgccaaagttgagaagggcaagagcaagaagcttaagtccgtcaaggaacttctgggcattactattatggagcgctctagctttgaaaagaatccgatcgacttcctcgaagccaaggggtacaaagaagtgaagaaggacctgataatcaaactgcctaagtacagtctgtttgagctcgagaacggccgcaaacggatgttggcttctgccggcgaactgcagaagggcaatgagttggcactgccctcaaagtacgtcaacttcctgtacttggcttcccattacgagaagcttaaggggagccctgaagataacgaacagaagcagctgttcgtcgagcagcacaagcactatctcgatgaaatcattgaacagatcagcgaattcagtaaaagggtcattctcgccgacgcaaatctggataaggtgctgagcgcctataacaagcacagggataaaccaattcgcgagcaagcagaaaacatcatccacctgttcactcttactaacctgggagcccccgccgccttcaaatacttcgacacaacaattgaccgaaaaagatacacatcaacaaaggaagtccttgacgctacactgattcaccagagcatcacaggtttgtacgaaacccggatcgatctgtcccagctggggggggac

(19) United States
(12) Patent Application Publication
(10) Pub. No.: US 2026/0132426 A1
(43) Pub. Date: May 14, 2026

(54) BOVINIZED CRISPR/BOCAS9 GENE EDITING SYSTEM, METHOD AND USE

(71) Applicant: INNER MONGOLIA UNIVERSITY, Huhhot, Inner Mongolia (CN)

(72) Inventors: Lei YANG, Huhhot, Inner Mongolia (CN); Zhenting HAO, Huhhot, Inner Mongolia (CN); Guangpeng LI, Huhhot, Inner Mongolia (CN); Chunling BAI, Huhhot, Inner Mongolia (CN); Xuefei LIU, Huhhot, Inner Mongolia (CN); Guanghua SU, Huhhot, Inner Mongolia (CN)

(21) Appl. No.: 19/131,646

(22) PCT Filed: Jan. 22, 2024
(86) PCT No.: PCT/CN2024/073497

§ 371 Date: May 21, 2025

(30) Foreign Application Priority Data
May 11, 2023 (CN) 202310524020.8

(51) Int. Cl. C12N 15/90; C12N 9/22; C12N 15/11

(52) U.S. Cl. CPC C12N 15/907; C12N 9/226; C12N 15/11; C12N 2310/20

(57) ABSTRACT

Provided is a bovinized CRISPRboCas9 gene editing system, comprising a boCas9 protein and sgRNA. The mucleotide sequence encoding the boCas9 protein is as shown in SEQ ID NO. 1. Further provided are a bovinized CRISPRboCas9 gene editing method, and the use. SEQ ID NO. 1 is obtained by means of optimizing an exogenous Cas9 gene according to the codon preference of bovine cells, so that the transcription and translation efficiency of the Cas9 gene in bovine cells can be significantly improved, and the content of Cas9 protein in the cells is finally increased. Results show that the expression level of the modified bovinized boCas9 protein is twice that of a wild-type control group Cas9, and the target gene cleavage efficiency is at least 6 times that of a wild-type control group.

### TECHNICAL FIELD

The invention relates to the technical field of genetic engineering, in particular to a bovinized CRISPRboCas9 gene editing system.

### BACKGROUND ART

The CRISPR/Cas9 system is an acquired immune system found in bacteria and archaea, comprising two core components: sgRNA and Cas9 protein. The sgRNA can guide the Cas9 protein to specific target regions, while Cas9 functions as a nuclease capable of cleaving double-stranded DNA. Among all CRISPR/Cas9 systems, the most commonly used is the SpCas9 optimized from Streptococcus pyogenes. SpCas9 has become the most widely used gene-editing technology because it possesses the simplest PAM sequence (5′-NGG-3′), enabling editing of nearly all gene sequences.

Proteins in organisms consist of 20 amino acids encoded by 64 codons, with each amino acid corresponding to at least one codon. Significant differences exist in codon usage frequency across different organisms, showing strong preference—frequently used codons are termed optimal codons while rarely used ones are called rare codons. When the coding sequence of exogenous genes aligns more closely with the codon preference of target cells, the protein translation efficiency of exogenous genes increases. Therefore, when employing the CRISPR/Cas9 gene editing system in bovine cells, codon preference optimization of the Streptococcus pyogenes-derived Cas9 protein has been implemented to establish a high-efficiency targeting bovinized CRISPR/Cas9 system, which provides an effective technical means for bovine genome targeting editing and biological breeding modification research.

### SUMMARY OF THE INVENTION

For this purpose, the invention provides a bovinized CRISPR/boCas9 gene editing system, method, and use.

To achieve the above objectives, embodiments of the present invention provide the following technical solutions:

According to the first aspect of the embodiments of the invention, a bovinized CRISPR/boCas9 gene editing system is provided, comprising a boCas9 protein and sgRNA. The mucleotide sequence encoding the boCas9 protein is as shown in SEQ ID NO.1.

Furthermore, the sgRNA is selected from any one of sgRNA1-5, wherein:
the nucleotide sequence of a sense strand of sgRNA1 is shown in SEQ ID NO.2, and the nucleotide sequence of an antisense strand is shown in SEQ ID NO.3;
the nucleotide sequence of a sense strand of sgRNA2 is shown in SEQ ID NO.4, and the nucleotide sequence of an antisense strand is shown in SEQ ID NO.5;
the nucleotide sequence of a sense strand of sgRNA3 is shown in SEQ ID NO.6, and the nucleotide sequence of an antisense strand is shown in SEQ ID NO.7;
the nucleotide sequence of a sense strand of sgRNA4 is shown in SEQ ID NO.8, and the nucleotide sequence of an antisense strand is shown in SEQ ID NO.9;
the nucleotide sequence of a sense strand of sgRNA5 is shown in SEQ ID NO.10, and the nucleotide sequence of an antisense strand is shown in SEQ ID NO.11.

According to the second aspect of the invention, a gene editing method using the aforementioned bovinized CRISPR/boCas9 gene editing system is provided, comprising the following steps:
(1) constructing a boCas9 expression vector and sgRNA expression vector separately;
(2) co-transfecting the boCas9 expression vector and sgRNA expression vector into bovinized cells;
(3) detecting the cleavage efficiency of target genes in the bovinized cells.

Furthermore, the boCas9 gene sequence is constructed on a pSpCas9 vector to obtain a pboCas9 expression vector.

Furthermore, target genes are MSTN, Rosa26, HEBP1, LDLR, or PRNP.

Furthermore, the T7E1 enzymatic digestion method is used for cleavage efficiency detection.

According to the third aspect of the invention, a use for the aforementioned bovinized CRISPR/boCas9 gene editing system is provided in editing mammalian DNA and transgenic breeding.

Furthermore, the editing includes genome-targeted excision, insertion, or modification.

Design thought of the invention: with the rapid development of gene editing technology and molecular breeding, there is an urgent need for a mature gene editing system for bovine-derived somatic cells and embryonic cells. Due to codon preference, the Streptococcus pyogenes-derived Cas9 protein exhibits low gene editing cleavage efficiency in bovine cells and cannot efficiently complete target gene cleavage. Therefore, codon preference optimization of the Streptococcus pyogenes-derived Cas9 gene for bovine cells has been implemented to establish a high-efficiency bovinized CRISPR/boCas9 gene editing system. This serves as foundational technology for gene knockout, gene insertion, and site-specific gene modification.

The invention has the following advantages:
the invention establishes a high-efficiency bovinized CRISPR/boCas9 gene editing system. This system involves codon optimization of the Streptococcus pyogenes-derived SpCas9 protein for bovine cells, followed by detection of the cleavage efficiency of the optimized CRISPR/boCas9 gene editing system and comparison of its cleavage efficiency across different target sites. Results demonstrate that the cleavage efficiency of the bovinized CRISPR/boCas9 gene editing system is at least 6 times that of the conventional CRISPR/Cas9 gene editing system. This advancement provides an effective technical means for research on site-specific genome excision, insertion, and modification, as well as for transgenic breeding studies.

### BRIEF DESCRIPTION OF ACCOMPANY DRAWINGS

To more clearly illustrate the embodiments of the invention or technical solutions in the prior art, the following will briefly introduce the drawings required for describing the embodiments or the prior art. It is evident that the drawings in the following description are exemplary only. For those of ordinary skill in the art, other implementation drawings may be derived based on the provided drawings without creative effort.

FIG. 1 is a flowchart illustrating the construction method of the CRISPR/boCas9 gene editing system provided by the invention.

FIG. 2 is a comparative diagram showing the cleavage efficiency of the CRISPR/boCas9 gene editing system versus the CRISPR/SpCas9 gene editing system on different target genes.

FIG. 3 is a comparison diagram of Cas9 protein expression levels between the CRISPR/boCas9 gene editing system provided by the invention and the CRISPR/LwCas9 gene editing system.

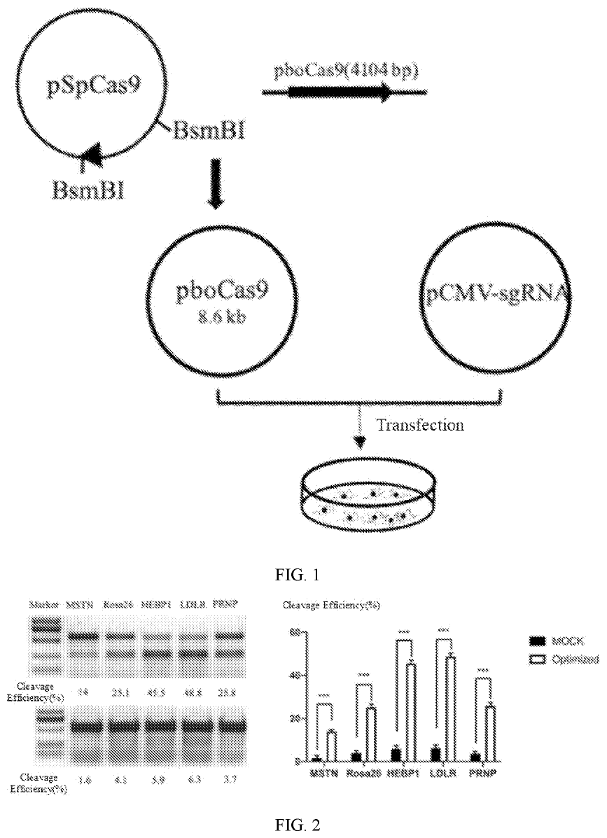

### SPECIFIC EMBODIMENT OF THE INVENTION

The implementation of the invention is described below through specific embodiments. Those skilled in the art may readily understand other advantages and effects of the invention from the content disclosed in this specification. It is evident that the described embodiments represent only a portion of the invention's implementations and not all possible embodiments. All other embodiments obtained by persons of ordinary skill in the art based on the embodiments herein without creative effort shall fall within the scope of protection of the invention.

In the following embodiments, the experimental materials and reagents used include:

T4 DNA Ligase is purchased from Thermo Scientific; pSpCas9 vector is obtained from Addgene; pCMV-sgRNA plasmid is acquired from Origene; DMEM medium, Opti-MEM medium, and fetal bovine serum (FBS) are purchased from Gibco; Lipofectamine 3000 transfection reagent is procured from Invitrogen.

In the following embodiments, molecular biology experimental methods not specifically described were performed according to the specific protocols outlined in Molecular Cloning: A Laboratory Manual or followed the instructions provided with the reagent kits and product manuals.

### Embodiment 1: Construction of a Bovinized CRISPR/boCas9 Gene Editing System

1. Prediction of the DNA sequence for the SpCas9 protein in bovine-derived cells was conducted based on the known SpCas9 DNA sequence, including the following steps:
(1) DNA sequence optimization of the SpCas9 gene in bovine-derived cells was performed using the website https://www.novopro.cn/. The optimized boCas9 nucleotide sequence is as follows, wherein uppercase letters indicate optimized bases:

(SEQ ID NO. 1)
atggaCaagaaGtaTtcCatCggcCtGgaCatAggAacCaaCTCTgtGggTtgggcCgtTatcacCgatga
GtaCaaggtGccCtcCaaGaagttcaaAgtGctgggTaaCacCgaTAgGcaTagCatTaaGaaaaatctGatCgggg
cATtGCtGtttgaTTCCggagaAacagcAgaGgcTactAgActcaaGAgAacTgcCAgGCgGCggtaCacT
AgGcgCaagaatAgGatCtgCtatctGcaAgagatCttttcCaaCgagatggcCaaGgtTgatgaCTCtttcttCcaCc
gaTtGgaGgagAGttttCtggtggaagaGgacaaAaagcaCgaGcgGcaCccAatCttCggaaacatTgtTgaCga
GgtGgcCtaCcaCgagaaataCccCacCatAtaCcatctTcgCaaaaaaCtggtCgatAGtacGgaCaaGgcCgaC
CtTAgACtTatctaCCtggcTCtTgcCcatatgatCaaAtttAgGggAcaCttCttgatCgagggagatCtCaaCcc
GgaCaaCagtgatgtggacaaGctGttCatTcagCtggtTcaGacctaTaaCcaattGtttgaGgaGaaccctatCaacgc
TTCAggCgtGgaCgctaaggcTatActCtctgcGcgGCtCTCAaaatcaCgacgGttGgaaaatctcatCgctcagc
tcccAggCgaAaagaaGaaCggTCtGttCggTaaCctGattgcCCtgAGTCtgggCCtCacAccGaatttCaaG
AGCaaCttCgaCCtCgcagaGgaCgcCaaGCtCcagctGtcCaaGgaCacCtaTgatgatgaCCtGgataatCt
GCtCgcCcaGatCggGgatcaGtatgcCgatCtgtttCtggcCgcCaaAaaCCtGtcTgaCgctatACtGctGAG
TgaCatTctGCgGgtGaaCacCgaGatTacAaaggcCccActTagcGCcagtatgatCaaaAgGtacgaCgaGca
CcatcaGgacCtgacActGttGaaGgcCCtGgtGAgacaGcaactCccCgaaaagtaCaaggaGatctttttCgatcaa
tcaaaaaacggCtaCgcTggGtatattgaCggAggCgcAagccaGgaagaGttCtaCaaGttCatcaaaccGattCtGg
aGaaaatggaCggCacCgaAgaGCtGCtTgtgaaGctGaatAgAgaagaCCtCctTAgGaagcaGcgcacTttC
gacaacggGAGCatCccGcaCcaGatAcaTCtCggGgagctgcatgctatCttgagGagGcaGgaagaTttttaCcc
GttCCtGaaGgacaaCcgCgaAaagatCgaGaaGatACtgacGttCAgGatAccCtaCtatgtGggGccTCtgg
cTcgAggAaatagCcgCttCgcGtggatgactAgAaaAtcAgaGgaaacaatAaccccCtggaattttgaagaGgtGg
tcgaCaaGggGgcGtcTgcCcaGAGCttCattgaGcgcatgacaaacttCgaCaaaaatctGccTaaCgaaaaagtTc
tGccTaaacaCTCtCtTctGtaCgagtatttCacTgtttataacgaaCtCacaaaggtGaaGtatgtGacCgaaggCatg
AgaaaaccCgcGtttctGtcTggGgaacagaagaaGgccatCgttgaCttGctGttTaaGacCaatcgCaaGgtCacG
gtGaagcaGCtGaaGgaGgaCtaCttcaaaaaGatagaGtgtttCgaCagCgtGgaaatCtcCggagtCgaagaCCg
GtttaaCgcGAGTCtGggGacctaccaCgaCCtgctGaaGattatCaaGgaCaaGgaCtttCtCgaCaaCgaGga
aaaCgaGgatatACtGgaggaCatCgtGttGacCCtgacGCtGttCgaGgatCgggagatgatCgaggaGCgGct
GaaGacTtatgcGcacctGtttgatgaCaaggtTatgaaGcagctCaaGAgAAgAcgGtaCacCggCtggggCcg
GCtTtcCAgaaaGCtCatCaatggAatCagggataaAcaGtctggTaaGacaatCttGgatttCCtgaaGAGCgatg
gGttCgcTaaCcgcaatttCatgcagctgatTcaCgatgatTCtCtCacattCaaagaagaTattcaGaaGgcCcaagtCtc
CggacaGggcgatTCtCtGcaCgaacaCatCgcCaatCtTgcGggGTCccctgcCatCaaaaaGggAattCtTca
gactgtCaaGgtCgtGgaCgaGCtggtGaaagtGatgggCcggcaCaagccTgaaaatatAgtGattgaGatggcaAg
AgaaaaCcaAacaactcaGaaaggccagaaGaattcAcgagaAAgGatgaaGcgGatAgaGgaaggCatTaaGga
GCtGggCTCCCaAattctGaaagagcatccCgtGgaGaaCacAcaGCtCcaGaaCgaGaagctTtaCTtGtaCt
aCTtGcaGaaCggCagagacatgtaCgtggaTcaGgaGCtGgaCatCaatcgattGTCAgaCtatgaCgtGgatca
catCgttccacaGagCttcctGgcAgacgattcCatCgaTaaCaaggtGCtTacTcgGtcCgaCaaGaaCcgGggGa
aGAGTgataaTgtCccCagCgaGgaGgtGgtGaaGaagatgaaGaaTtaCtggagacaGTtGctGaaTgcGaag
CtCatcacCcaGcgCaagtttgataaCCtTacAaaagcCgaaAgAggGggAttgTCCgaaTtGgaCaaGgcCgg
AtttatTaaGAgAcaGCtTgtCgaGacCcgccaGatcacAaagcaCgtCgcTcaaatACtggatTCCcgcatgaata
cCaaGtacgatgaaaaCgataaGctGatCcgGgaAgtGaaGgtgattaccCtGaaatctaaattGgtGAGtgacttTAg
GaaGgatttccaGttctataaagtGcgAgaAatAaaTaaCtaTcatcaCgcGcatgatgcCtatctGaaCgcAgtcgtGg
gCacCgcACtgatAaagaaataCccCgcCctGgaGAGCgagtttgtGtaCggCgaCtataaGgtGtatgaCgtGA
gaAaaatgattgcCaaAtcCgaAcaGgaGatCggTaaGgcTacTgcTaaatatttTttCtactcAaaCatcatgaacttTt
tcaaGacGgaaattacCTtGgcTaatggCgaAatCcgAaaagcAccCctTatTgaGacCaatggggaaacGggCga
aatAgtGtgggataaGggCAgagattttgcAacCgtgAgGaaagtCCtCAGcatgccccaagtGaaCatCgtGaaAa
aGacagaGgtTcaAacTggGggattctccaaAgagtcCattCtGccGaaGagaaaCAGTgaTaaActGatCgcGc
gCaaaaaGgactgggatccGaaGaaGtaCggGggCtttgatTCtccTacTgtTgcGtaCAGCgtGctCgtCgtCgc
CaaAgtTgaGaaGggCaaGAGCaagaagCtTaaGtccgtCaaGgaACtTctGggCatTacTattatggaGCgC
TCtAGctttgaaaaGaatccgatCgacttCCtCgaagcCaaGggGtaCaaAgaagtGaaGaaGgacCtGatAatCa
aactGcctaaGtaCagtctGtttgagCtCgaGaacggCcgCaaacggatgTtggctTCtgccggCgaaCtGcaGaaGg
gCaatgagTtggcActgccCTCAaaGtaCgtCaaCttCCtGtaCttGgctTCCcattaCgaGaagCtTaagggGag
CccTgaagataacgaacaGaaGcaGCtgttCgtcgagcagcaCaagcaCtatCtCgatgaAatCattgaAcaGatcagC
gaattCAGtaaAAgGgtCattCtCgcCgaCgcAaatCtGgataaGgtGctGagCgcCtataacaaGcaCagGgaT
aaaccaatTcgCgaGcaagcagaaaaCatCatCcaCCtGttCacTCtTacTaaCctGggagcCcccgcCgcCttCaa
ataCttCgaCacaacaattgaCcgAaaaAgataCacAtcAacaaaGgaagtCCtTgaCgcTacActGatTcaCcaG
AGcatcacAggtTtGtaCgaaacCcgGatCgatCtgTCCcagctGggGggGgac.

(2) The optimized sequence was submitted to BGI Group for synthesis. Subsequently, the pSpCas9 vector was linearized using BsmB I enzyme digestion. The synthesized optimized sequence was then ligated into a linearized vector with T4 DNA ligase and sent to a sequencing company for validation.
2. Construction method for sgRNA sequence vectors of targeting genes, comprising the following steps:
(1) five genes (MSTN, Rosa26, HEBP1, LDLR, and PRNP) were selected as target genes. Their respective sgRNAs were synthesized and cloned into a pCMV-sgRNA vector. The sgRNA sequence for the target genes are as follows:

sense strand for sgRNA1 of MSTN gene is:
(SEQ ID NO. 2)
CAAAGTTGGTGACGTGACAGAGG,
while the antisense strand thereof is:
(SEQ ID NO. 3)
CCTCTGTCACGTCACCAACTTTG;
sense strand for sgRNA2 of Rosa26 gene is
(SEQ ID NO. 4)
GGGACCCGAGCCAATAACAA,
while the antisense strand thereof is:
(SEQ ID NO. 5)
TTGTTATTGGCTCGGGTCCC;
sense strand for sgRNA3 of HEBP1 gene is:
(SEQ ID NO. 6)
TAGGTTCCCATCGTCACT,
while the antisense strand thereof is:
(SEQ ID NO. 7)
CAGTGACGATGGGAACCTA;
sense strand for sgRNA4 of LDLR gene is:
(SEQ ID NO. 8)
GCCATTGTGGTCGATCC,
while the antisense strand thereof is:
(SEQ ID NO. 9)
GGATCGACCACAATGGC;
sense strand for sgRNA5 of PRNP gene is:
(SEQ ID NO. 10)
GGAAGCCCTCCTGCCGCAAC,
while the antisense strand thereof is:
(SEQ ID NO. 11)
GTTGCGGCAGGAGGGCTTCC;

(2) a 5′-end of sgRNA sense oligonucleotide sequence of the target gene is added with AGGAG, and a 3′-end is added with G to form a sequence of a sticky end; adding an AAAC sequence at a 5′-end and adding a C at a 3′-end of the sgRNA antisense oligonucleotide sequence of the target gene; the synthesized sense oligonucleotide sequence and antisense oligonucleotide sequence were annealed and renatured to form double-stranded oligonucleotides with sticky ends. The procedure is detailed in Table 1.

TABLE 1
Annealing Procedure

| Procedure | Time |
|---|---|
| 37° C. | 5 min |
| 95° C. | 10 min |

−5° C./min

| | |
|---|---|
| 4° C. | ∞ |

(3) The pCMV-sgRNA vector was linearized by digestion with BsmBIrestriction enzyme. The digestion system is detailed in Table 2.

TABLE 2
Digestion System

| Reagent | Volume |
|---|---|
| BsmBI | 1 μl |
| pCMV-sgRNA plasmid | 1 ug |
| Cutsmart Buffer (10×) | 5 μl |
| ddH₂O | Up to 50 μl |

The target gene was ligated to the linearized vector, with a ligation system as detailed in Table 3.

TABLE 3
Ligation System

| Reagent | Volume |
|---|---|
| pCMV-sgRNA | 2 μl |
| sgRNA | 1 μl |
| Solution 1 | 5 μl |
| ddH₂O | Up to 10 μl |

After ligation at 16° C. for 3 hours, the product was transformed into DH5a competent cells. Single colonies were picked and sent for sequencing to obtain a recombinant expression vector pCMV-sgRNA of the target gene sgRNA oligonucleotides. shake culture was performed for correct positive clones, and plasmids were extracted.
3. The optimized pboCas9 expression vector and pCMV-sgRNA expression vector were co-transfected into bovine muscle satellite stem cells. Positive cells were collected, and cell genome was extracted, Using DNA of the cell genome as a template to amplify gene fragments containing the above target sequences. The amplified products were then subjected to T7E1 enzymatic digestion verification to determine the knockout efficiency of the target gene.

### Embodiment 2: Detection of Cleavage Efficiency for the Bovinized CRISPR/boCas9 Gene Editing System

1. The cleavage efficiency of the bovinized CRISPR/boCas9 system was detected using the T7E1 enzymatic digestion for verifying, comprising the following steps:
(1) a subset of target cells was collected for extracting DNA of the cell genome, and DNA of wild-type target cell genome was extracted at the same time.
(2) DNA of the cell genome was used as a template to amplify gene fragments containing the target sequences. The amplification primer sequences are listed in Table 4.

TABLE 4
Amplification Primer Sequences

| Locus | Direction | Amplification Primer |
|---|---|---|
| MSTN | F | 5′-CACTCTTCTGGCTTATCT-3′ (SEQ ID NO. 12) |
| | R | 5′-GAGGCACAGACTCAGAAGAAGA-3′ (SEQ ID NO. 13) |
| Rosa26 | F | 5′-CAACTCGCTGCCAATCAGC-3′ (SEQ ID NO. 14) |
| | R | 5′-TCGAGCTGGGTAGCCTTAATTG-3′ (SEQ ID NO. 15) |
| HEBP1 | F | 5′-GAAAGAAGAATGAGACCAGCAGATG-3′ (SEQ ID NO. 16) |
| | R | 5′-GTGGGAGATACAGGTAAAGGTGGTC-3′ (SEQ ID NO. 17) |
| LDLR | F | 5′-TCTTTACCAACCGCCACGAA-3′ (SEQ ID NO. 18) |
| | R | 5′-ATTCCCACCATGACGGAACC-3′ (SEQ ID NO. 19) |
| PRNP | F | 5′-ACAGTCGGGTATACCAGTTG-3′ (SEQ ID NO. 20) |
| | R | 5′-TCAATGGGTGTTGTCACCAG-3′ (SEQ ID NO. 21) |

(3) The amplified product was treated with T7E1 enzyme and subjected to denaturation again and programmed gradient annealing, the procedure is shown in Table 5.

TABLE 5
Denaturation and Annealing Procedures

| | |
|---|---|
| Temperature | Time |

| | | | |
|---|---|---|---|
| 95° | C. | 5 | min |
| 95~85° | C. | −2° | C./sec |
| 85~25° | C. | −0.1° | C./sec |

| | | |
|---|---|---|
| 4° | C. | ∞ |

Mutant and wild-type DNA single strands randomly anneal to form new double stranded DNA. The nucleic acid electrophoresis results were analyzed using ImageJ grayscale analysis software, and the cleavage efficiency f_{cut} was calculated with the following formula:

$${f}_{cut} = ( sum of grayscale values of two post - digestion bands ) / ( total grayscale value of all three bands in the lane )$$

To accurately determine the cleavage efficiency, the cleavage efficiency f_{cut} was converted to the actual mutation rate (Indel %) using the formula:

$$Indel \% = ( 1 - \sqrt{1 - fcut} ) \times 100$$

FIG. 2 is a comparative diagram showing the cleavage efficiency of the CRISPR/boCas9 gene editing system versus the CRISPR/SpCas9 gene editing system. The left picture (upper part) displays the T7E1 cleavage activity result of the CRISPR/boCas9 gene editing system, while the left picture (lower part) shows the T7E1 cleavage activity result of the CRISPR/SpCas9 gene editing system. The right picture is a quantitative statistical graph of the cleavage efficiency of the CRISPR/boCas9 gene editing system (Optimized) and the CRISPR/SpCas9 gene editing system (Mock). Table 6 summarizes the cleavage efficiency comparison results between the CRISPR/boCas9 gene editing system and the CRISPR/SpCas9 gene editing system.

TABLE 6
Cleavage Efficiency Comparison Results

| | MSTN | Rosa26 | HEBP1 | LDLR | PRNP |
|---|---|---|---|---|---|
| CRISPR/SpCas9 | 1.6 | 4.1 | 5.9 | 6.3 | 3.7 |
| gene editing system | | | | | |
| CRISPR/boCas9 | 14 | 25.1 | 45.5 | 48.8 | 25.8 |
| gene editing system | | | | | |

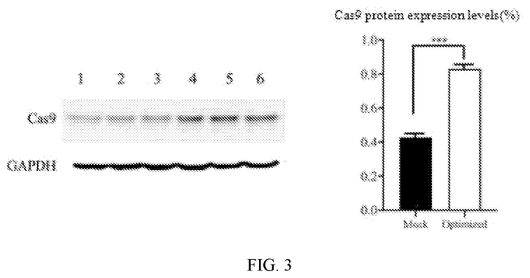

As shown in Table 6 and FIG. 2, the cleavage efficiency of the optimized CRISPR/boCas9 gene editing system is at least 6 times that of the CRISPR/SpCas9 system before optimization.
2. Western Blot verification was used to determine the Cas9 protein expression level in the bovinized CRISPR/boCas9 gene editing system. The steps are as follows: cultured cells were gently washed twice with PBS. Each sample was added with 1 mL of protein lysis buffer (990 μL RIPA+10 μL PMSF), mixed thoroughly, incubated at 4° C. for 30 minutes, and centrifuged at 12,000 rpm and 4° C. for 30 minutes, and the supernatant is protein sample, stored at −80° C., western blotting was subsequently performed using total protein extracted before and after the optimization. FIG. 3 is a comparison diagram of Cas9 protein expression levels between the CRISPR/boCas9 gene editing system and the CRISPR/SpCas9 gene editing system, wherein, top bands 1-3 in the left picture is SpCas9 protein in the CRISPR/SpCas9 gene editing system, top bands 4-6 in the left picture is bovinized boCas9 protein of the CRISPR/boCas9 system; while bottom bands GAPDH in the left picture is loading control protein, and the right picture shows a quantitative statistical comparison of Cas9 protein expression levels between the CRISPR/boCas9 system (Optimized) and the CRISPR/SpCas9 system (Mock). The results demonstrate that the protein expression levels of CRISPR/boCas9 system (Optimized) is twice that of the CRISPR/SpCas9 system (Mock).

This study demonstrates that the optimized CRISPR/boCas9 system is a highly efficient gene editing system in bovine cells, providing a robust technical platform for targeted genome excision, insertion, modification, and transgenic breeding research.

Although the invention has been described in detail with general explanations and specific embodiments, modifications or improvements apparent to those skilled in the art shall fall within the scope of protection of the present invention, provided they do not depart from its spirit.

1. A gene editing method using a bovinized CRISPR/boCas9 gene editing system, comprising the following steps:
(1) constructing a boCas9 expression vector and sgRNA expression vector separately;
(2) co-transfecting the boCas9 expression vector and sgRNA expression vector into bovinized cells;
(3) detecting the cleavage efficiency of target genes in the bovinized cells;
the bovinized CRISPR/boCas9 gene editing system comprising a boCas9 protein and sgRNA; the mucleotide sequence encoding the boCas9 protein is as shown in SEQ ID NO.1; the sgRNA is selected from any one of sgRNA1-5, wherein:
the nucleotide sequence of a sense strand of sgRNA1 is shown in SEQ ID NO.2, and the nucleotide sequence of an antisense strand is shown in SEQ ID NO.3;
the nucleotide sequence of a sense strand of sgRNA2 is shown in SEQ ID NO.4, and the nucleotide sequence of an antisense strand is shown in SEQ ID NO.5;
the nucleotide sequence of a sense strand of sgRNA3 is shown in SEQ ID NO.6, and the nucleotide sequence of an antisense strand is shown in SEQ ID NO.7;
the nucleotide sequence of a sense strand of sgRNA4 is shown in SEQ ID NO.8, and the nucleotide sequence of an antisense strand is shown in SEQ ID NO.9;
the nucleotide sequence of a sense strand of sgRNA5 is shown in SEQ ID NO. 10, and the nucleotide sequence of an antisense strand is shown in SEQ ID NO.11.

2. The gene editing method according to claim 1, wherein the boCas9 gene sequence is constructed on a pSpCas9 vector to obtain a pboCas9 expression vector.

3. The gene editing method according to claim 1, wherein target genes are MSTN, Rosa26, HEBP1, LDLR, or PRNP.

4. The gene editing method according to claim 1, wherein the T7E1 enzymatic digestion method is used for cleavage efficiency detection.

5. A use for the bovinized CRISPR/boCas9 gene editing system according to claim 1 in editing bovine-derived cell DNA.

6. A use according to claim 5, wherein the editing includes genome-targeted excision, insertion, or modification.
